(19) United States
(12) Patent Application Publication
(10) Pub. No.: US 2025/0207704 A1
(43) Pub. Date: Jun. 26, 2025

(54) CONNECTOR COUPLING ASSEMBLY WITH RELIEF CUTS

(71) Applicant: CareFusion 303, Inc., San Diego, CA (US)

(72) Inventors: Leah Paige Gaffney, Orange, CA (US); Siddarth Shevgoor, Mission Viejo, CA (US); Seyed Alireza Salehi Borojerdi, Aliso Viejo, CA (US); Mohammed Mehtab Khan, Bengaluru (IN); Kuochu Colin Huang, Walnut, CA (US); Jason Andrew Wine, Brea, CA (US)

(21) Appl. No.: 18/390,553

(22) Filed: Dec. 20, 2023

(51) Int. Cl. F16L 37/46; A61M 39/10

(52) U.S. Cl. CPC F16L 37/46; A61M 39/10; F16L 2201/20

(57) ABSTRACT

A coupler including a first connector having a first end, a second end opposite the first end, and a valve disposed between the first end and the second end. The second end including a mating portion, wherein the valve extends at least partially into the mating portion. The coupling including a second connector having a housing, a plurality of flanges extending from the housing, each of the plurality of flanges being separated from an adjacent flange by a cut. The plurality of flanges configured to engage with the mating portion when the second connector is coupled to the first connector. The first connector is configured to decouple from the second connector in response to a pullout force exceeding a predetermined threshold force.

### FIELD OF THE INVENTION

The present disclosure generally relates to connectors, and, in particular, to connector couplings having relief cuts.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. Often, tubing or catheters are coupled or secured to each other to allow fluid communication between various portions of tubing or catheters.

In some applications, such tubing or catheters may become dislodged due to improper securement and/or when the coupling is subject to forces greater than what the coupling is designed to withstand.

### SUMMARY

One or more embodiments of the present invention are directed to a coupler including a first connector having a first end, a second end opposite the first end, and a valve disposed between the first end and the second end, the second end including a mating portion, wherein the valve extends at least partially into the mating portion, and a second connector having a housing, a plurality of flanges extending from the housing, each of the plurality of flanges being separated from an adjacent flange by a cut, the plurality of flanges configured to engage with the mating portion when the second connector is coupled to the first connector. The first connector is configured to decouple from the second connector in response to a pullout force exceeding a predetermined threshold force.

In some embodiments, the mating portion includes a groove circumferentially disposed on the mating portion, the groove configured to receive a portion of one of the plurality of flanges. Each of the plurality of flanges includes a ridge, the ridge configured to engage the groove when the first connector is coupled to the second connector.

In some embodiments, the second connector includes three cuts and three flanges.

In some embodiments, the pullout force is a force applied to the second connector along a central axis of the second connector and the central axis extends at least along a length of the second connector. The central axis extends through the first connector and the second connector when the first connector is coupled to the second connector.

In some embodiments, the valve is configured to extend at least partially into the housing when the first connector is coupled to the second connector.

In some embodiments, the coupler has a first configuration and in the first configuration the first connector is coupled to the second connector such that the valve is at least partially disposed within the housing.

In some embodiments, the coupler has a second configuration and in the second configuration the first connector is disconnected from the second connector.

In some embodiments, the first connector is coupled to a first portion of tubing at the first end and the second connector is coupled to a second portion of tubing at the connecting portion.

In some embodiments, the second connector includes a gap disposed between the plurality of flanges and the housing and the mating portion is at least partially disposed within the gap when the first connector is coupled to the second connector.

In some embodiments, a fluid pathway is formed between the second connector and the first connector when the first connector is coupled to the second connector.

In some embodiments, the housing includes a plurality of ribs circumferentially disposed around the housing, the plurality of ribs configured to contact the mating portion when the first connector is coupled to the second connector.

In some embodiments, the housing includes a ring circumferentially disposed around the housing, the ring configured to contact the mating portion when the first connector is coupled to the second connector.

In some embodiments, the plurality of flanges are biased radially inward and are configured to flex radially outward when coupling the first connector to the second connector. he plurality of flanges form a ring, the ring being concentric with an opening of the housing. The plurality of flanges are configured to deflect away from a central axis when decoupling the first connector to the second connector.

In some embodiments, the mating portion is at least partially disposed within the second connector when the first connector is coupled to the second connector.

One or more embodiments of the present invention are directed to a coupler including a first connector having a first end, a second end opposite the first end, a mating portion disposed proximate the second end, the first connector including a valve disposed between the first end and the second end, the valve at least partially extending into the mating portion, and a second connector having a housing and ring extending from the housing, the ring having a plurality of cuts forming a plurality of flanges, each of the plurality of flanges including a ridge extending radially inward and each of the plurality of flanges being biased radially inward. The first connector is configured to decouple from the second connector in response to a pullout force exceeding a predetermined threshold force. At least one of the plurality of flanges is configured to deflect radially outward to allow the first connector to decouple from the second connector when the pullout force exceeds the predetermined threshold force.

One or more embodiments of the present invention are directed to a coupler including a first connector having a first end, a second end opposite the first end, a tubing portion disposed proximate the first end and a mating portion disposed proximate the second end, the first connector including a valve disposed between the first end and the second end, the valve at least partially extending into the mating portion, the first connector including a groove circumferentially disposed around the mating portion, and a second connector having a housing and ring extending from the housing, the ring having a plurality of cuts forming a plurality of flanges, each of the plurality of flanges including a ridge extending radially inward and each of the plurality of flanges being biased radially inward, the second connector including a gap between the housing and the ring, the gap configured to receive a portion of the mating portion when the first connector is coupled to the second connector. A fluid pathway is formed between the first connector and the second connector when the first connector is coupled to the second connector. The first connector is configured to decouple from the second connector in response to a pullout force exceeding a predetermined threshold force. At least one of the plurality of flanges is configured to deflect radially outward to allow the first connector to decouple from the second connector when the pullout force exceeds the predetermined threshold force.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a system diagram showing a coupler assembly in use, in accordance with various aspects of the present disclosure.

FIG. 2 is a side view of the coupler assembly of FIG. 1, in accordance with various aspects of the present disclosure.

FIG. 3 is a cross sectional side view of the coupler assembly of FIG. 1.

FIG. 4A is a front perspective view of a second connector of the coupler assembly of FIG. 1, in accordance with various aspects of the present disclosure.

FIG. 4B is a front view of the second connector of FIG. 4A, in accordance with various aspects of the present disclosure.

FIG. 4C is a back perspective view of the second connector of FIG. 4A, in accordance with various aspects of the present disclosure.

FIG. 5A is an illustration of a computer simulation showing the deformation of the second connector of FIG. 4, in accordance with various aspects of the present disclosure.

FIG. 5B is an illustration of a computer simulation showing the deformation of the second connector of FIG. 4, in accordance with various aspects of the present disclosure.

FIG. 6A is a zoomed in cross-sectional view of the coupler assembly of FIG. 1 having a second connector with centering ribs, in accordance with various aspects of the present disclosure.

FIG. 6B is a side perspective view of the second connector of FIG. 6A, in accordance with various aspects of the present disclosure.

FIG. 7A is a side view of a second connector of having a bearing ring, in accordance with various aspects of the present disclosure.

FIG. 7B is a cross-sectional side view of the second connector of FIG. 7A, in accordance with various aspects of the present disclosure.

### DETAILED DESCRIPTION

The disclosed coupler assembly includes a first connector and a second connector. The first connector is configured to couple to the second connector. The coupler assembly may have a first configuration and a second configuration. In the first configuration, the first connector is coupled to the second connector. In the second configuration, the first connector is decoupled from the second connector.

The coupler assembly may be configured to couple a first portion of tubing to a second portion of tubing. For example, the first portion of tubing may be coupled to the first connector and the second portion of tubing may be coupled to the second connector. The first portion of tubing and/or the second portion of tubing may also couple to a patient or fluid source. In some embodiments, the coupler assembly allows for the flow of fluid from the first portion of tubing to the second portion of tubing. For example, when the first connector is coupled to the second connector, a fluid pathway may be formed through the first connector and the second connector to allow the flow of fluid from the first portion of tubing through the first connector and the second connector to the second portion of tubing. The fluid pathway may allow for the flow of fluid from the second portion of tubing through the second connector and the first connector to the first portion of tubing.

In some embodiments, the second connector is configured to decouple from the first connector. The second connector may be sterilized (e.g., via a sterilized cloth or a sterilizing device) or replaced with a new sterile connector to prevent infection or contamination that can occur if the second connector is re-used without sterilization. In some embodiments, the second connector is configured to decouple based on a force that exceeds a predetermined threshold force. When a force is applied to the second connector, such as a pullout force, that exceeds the predetermined threshold force, the second connector may decouple from the first connector. The pullout force may be a force that occurs along the longitudinal axis of the second connector and/or the coupler assembly. In some embodiments, the pullout force is caused by tugging or pulling on the second portion of tubing coupled to the second connector. Alternatively, the pullout out force applied to the second connector may be caused by tugging or pulling on the first connector and/or the first portion of tubing coupled to the first connector.

In some embodiments, once the first connector is decoupled from the second connector, the second connector is configured to be re-coupled to the first connector. For example, once the second connector decouples from the first connector, the first connector may be configured to allow for re-coupling to the second connector after a disconnection event.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for case of understanding. Reference numbers may have letter suffixes appended to indicate separate instances of a common element while being referred to generically by the same number without a suffix letter.

While the following description is directed to the connection of medical fittings for the administration of medical fluid using the disclosed coupler, it is to be understood that this description is only an example of usage and does not limit the scope of the claims. Various aspects of the disclosed coupler may be used in any application where it is desirable to secure the connection of various tubing and fittings.

The disclosed coupler assembly overcomes several challenges discovered with respect to certain conventional couplers. One challenge with certain conventional couplers is that certain conventional couplers may be improperly secured. Further, during use, certain conventional couplers may be designed to release or dislodge in response to relatively low pullout forces. For example, certain conventional couplers may release in response to pullout forces experienced during patients rolling over in bed, patients catching tubing or lines on bed rails, moving patients to a different bed, fidgeting by pediatric patients, and/or disoriented adult patients pulling out their lines. Indeed, the Association for Vascular Access (AVA) Annual Scientific Meeting in 2017 reported a 10% dislodgement rate for 1,000 patients fitted with peripheral IV catheters, translating to approximately 33 million dislodgements per year in the U.S. alone. Because the accidental or unintentional dislodgement of tubing, catheters, or fittings may interrupt the administration of medical fluids, the use of certain conventional couplers is undesirable.

Therefore, in accordance with the present disclosure, it is advantageous to provide couplers and coupler/connector assemblies as described herein that allows for improved securement of fittings or connectors. The disclosed couplers and coupler/connector assemblies are structured as described herein so as to permit the secure retention of the first connectors, while allowing for decoupling after a disconnection event.

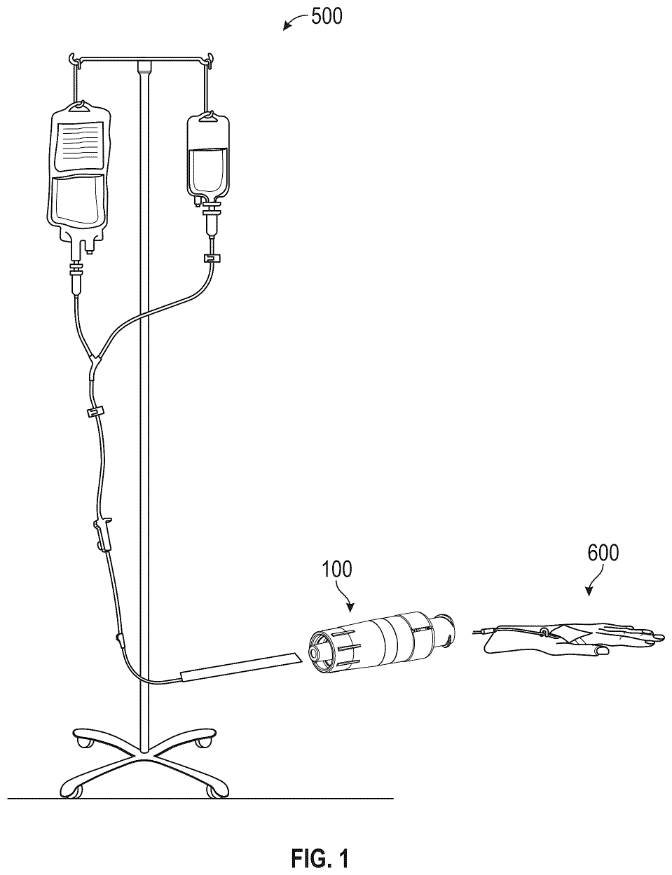

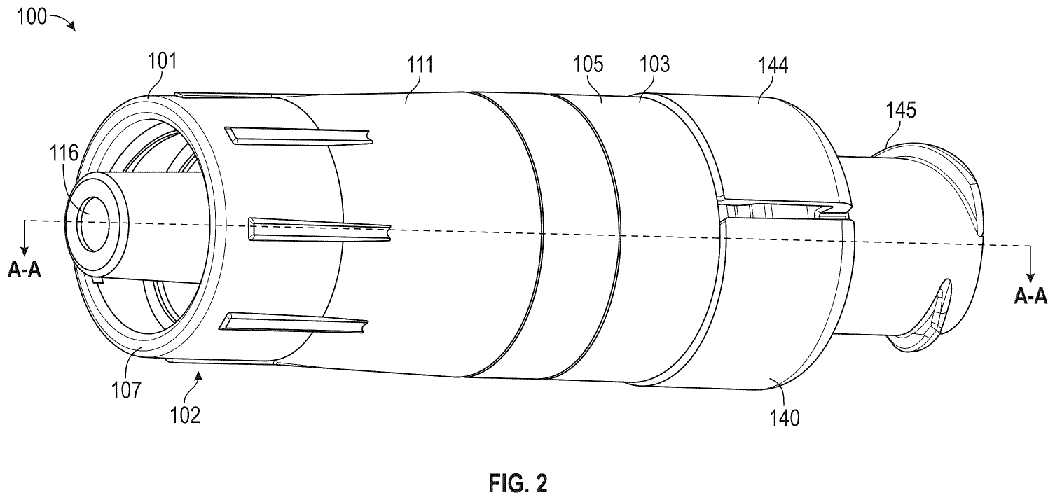

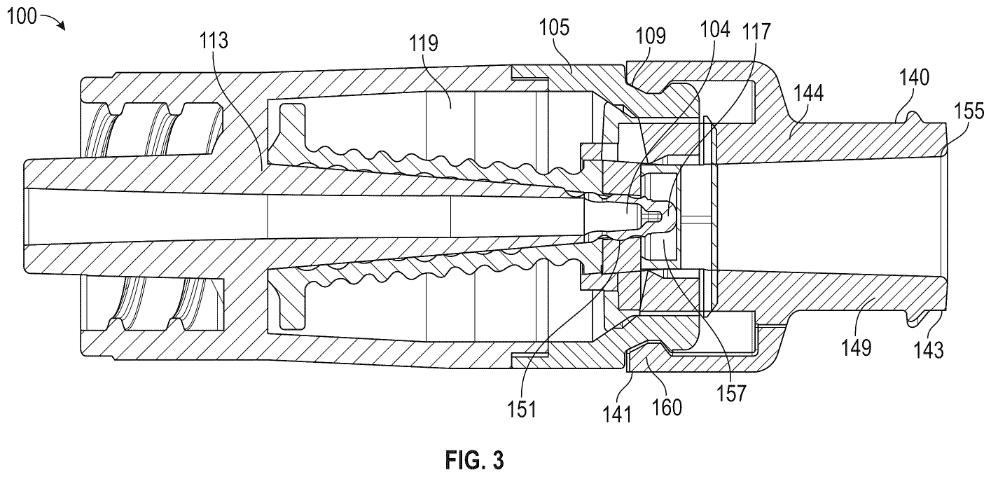

FIG. 1 is a system diagram showing a coupler assembly in use, in accordance with various aspects of the present disclosure. FIG. 2 is a side view of the coupler assembly of FIG. 1, in accordance with various aspects of the present disclosure. FIG. 3 is a cross sectional side view of the coupler assembly of FIG. 1.

With reference to FIGS. 1-3, coupler assembly 100 allows the flow of a fluid, such as a medical fluid, from fluid source 500 to patient end 600 by releasably coupling a portion of tubing or line with another portion of tubing or line in fluid communication. Coupler assembly 100 may include first connector 102, and second connector 140. First connector 102 may be configured to couple to second connector 140. In the depicted example, portions of tubing can be terminated with connectors/valves, such as first connector 102 and/or second connector 140. In some embodiments, fluid from fluid source 500 flows through coupler assembly 100 to patient end 600. A cannula or needle may be inserted within a patient at patient end 600 allowing medical fluid to flow from fluid source 500 through coupler assembly 100 and into a patient at patient end 600. In some embodiments, decoupling of first connector 102 from second connector 140 interrupts or prevents flow from fluid source 500 to patient end 600.

In some embodiments, coupler assembly 100 includes central axis A-A and first connector 102 and second connector 140 are coupled in series along central axis A-A. First connector 102 and/or second connector 140 may allow for the connection and/or disconnection of tubing to allow for selective fluid communication therebetween. Central axis A-A may extend longitudinally along the length of first connector 102 and second connector 140.

Coupler assembly 100 may have a first configuration and a second configuration. In the first configuration, first connector 102 is coupled to second connector 140. In the second configuration, first connector 102 is decoupled from second connector 140. In some embodiments, coupler assembly 100 transitions from the first configuration to the second configuration in response to a disconnection event. A disconnection even may occur when a pullout force is applied to second connector 140 or first connector 102. In some embodiments, a pullout force is applied to second connector 140 causing axial movement of second connector 140 relative to first connector 102. In some embodiments, axial movement of second connector 140 relative to first connector 102 is caused when the pullout force applied to second connector 140 exceeds a predetermined threshold force.

In some embodiments, first connector 102 is coupled to a first portion of tubing to allow the first portion of tubing to be connected and/or disconnected with second connector 140. First connector 102 may include first end 101 and second end 103. First end 101 may be coupled to tubing (e.g., a first portion of tubing) and second end 103 may be configured to couple to second connector 140. In some embodiments, a portion of tubing can be coupled with, or engage with first end 101 of first connector 102. First connector 102 via first end 101 may be in fluid communication with the tubing to allow fluid to pass through first connector 102. In some embodiments, first end 101 can have a flat surface to allow for clinicians to easily clean and disinfect first end 101. First end 101 may be in fluid connection with second end 103. First end 101 and second end 103 may be disposed along the longitudinal length of first connector 102. For example, first end 101 and second 103 may be disposed along central axis A-A. First end 101 and/or second end 103 may include an opening to allow first end 101 and/or second end 103 to be in fluid communication with one or more elements (e.g., tubing, connectors, valves, collars, attachments, etc.). For example, first end 101 may be coupled to a tube and second end 103 may include an opening to allow for fluid communication through first connector 102. In some embodiments, first connector 102 includes valve 104, which is configured to be disposed in first connector 102.

In some embodiments, fluid can exit or flow through first connector 102 via second end 103 disposed opposite to first end 101. The flow path through first connector 102 can have a straight fluid pathway to make flushing easier and to reduce the risk of hemolysis. Optionally, first connector 102 can include features (e.g., raised features, gripping features) disposed on the outer surface of first connector 102 to allow a clinician to more easily handle or manipulate first connector 102. Some embodiments of first connector 102 may provide connectors that are compatible with connectors of other portions of fluid delivery systems. First connector 102 may be substantially cylindrically shaped.

In some embodiments, first connector 102 includes tubing portion 107 and mating portion 105. Tubing portion 107 may be disposed at first end 101 and mating portion 105 may be disposed at second end 103. Tubing portion 107 may be configured to couple to a portion of tubing allowing first connector 102 to be in fluid communication with the portion of tubing. For example, tubing portion 107 may include channel 116 to allow for the flow of fluid within tubing portion 107. Channel 116 may be disposed within tubing portion 107 and extend the length of tubing portion 107.

In some embodiments, mating portion 105 is disposed opposite tubing portion 107 and second connector 140 is configured to couple to mating portion 105 to secure first connector 102 to second connector 140. For exampling mating portion 105 may include groove 109 configured to receive a portion of second connector 140 to secure second connector 140 to first connector 102, as described below. Groove 109 may be disposed on mating portion 105 proximate second end 103. In some embodiments, groove 109 is configured to receive and engage with a portion of second connector 140 to secure second connector 140 to first connector 102. Groove 109 may be circumferentially disposed around mating portion 105. Groove 109 may be continuously circumferentially disposed around mating portion 105. In some embodiments, groove 109 is circumferentially disposed around mating portion 105 in a repeated pattern or at predetermined locations.

In some embodiments, first connector 102 includes body 111 and valve 104. Body 111 may extend from tubing portion 107. For example, body 111 may extend away from tubing portion 107 towards second end 103. In some embodiments, body 111 and tubing portion 107 are formed of a unitary structure. Alternatively, body 111 may be fixedly or removably coupled to tubing portion 107. In some embodiments, body 111 includes channel 113. Channel 113 may be disposed within body 111 and may be in fluid communication with channel 116 of tubing portion 107. For example, tubing portion 107 may be coupled to fluid source 500 via a portion of tubing allowing fluid to flow from fluid source 500 and into channel 116. Fluid may flow from channel 116 into channel 113 of body 111. In some embodiments, body 111 is disposed between tubing portion 107 and mating portion 105. Body 111 may be coupled to tubing portion 107 and mating portion 105. In some embodiments, tubing portion 107, body 111, and mating portion 105 form a unitary structure.

In some embodiments, body 111 includes valve 104. Valve 104 may include distal end 117. Valve 104 may be disposed within body 111 and may be disposed proximate second end 103. In some embodiments, valve 104 is disposed between tubing portion 107 and second end 103. Valve 104 may be in fluid communication with channel 113, which may be in fluid communication with channel 116.

In some embodiments, when second connector 140 is couped to first connector 102, valve 104 at least partially extends into second connector 140. For example, when second connector 140 is couped to first connector 102, distal end 117 may be at least partially disposed within second connector 140. Valve 104 (e.g., distal end 117) being at least partially disposed within second connector 140 results in first connector 102 being in fluid communication with second connecter 140 when second connector 140 is couped to first connector 102. In other words, when second connector 140 is couped to first connector 102, a fluid pathway is formed between first connector 102 and second connector 140 via channel 113 and valve 104. In some embodiments, valve 104 is configured to be in an open position when second connector 140 is coupled to first connector 102. Valve 104 may be configured to be in a closed configuration when second connector 140 is decoupled from first connector 102. Valve 104 being in a closed configuration results in the fluid pathway through channel 113 and valve 104 being terminated preventing flow of fluid out of first connector 102. When second connector 140 is couped to first connector 102, valve 104 may be in the open position to allow fluid to flow from first connector 102, through channel 113 and valve 104, to second connector 140.

In some embodiments, a second portion of tubing is terminated by second connector 140 to allow the second portion of tubing to be connected and/or disconnected from first connector 102 Second connector 140 may include first end 141 and second end 143 disposed opposite first end 141. First end 141 may include housing 144 and second end 143 may include connecting portion 145, which may be disposed opposite housing 144. In some embodiments, a portion of tubing is coupled with, or engage with connecting portion 145 of second connector 140. In some embodiments, connecting portion 145 includes a threaded connection to facilitate coupling with tubing. For example, connecting portion 145 may include connecting portion 145 configured to couple to a portion of tubing.

In some embodiments, housing 144 includes features (e.g., threads) that allow for second connector 140 to mate with first connector 102. Housing 144 may fit together or otherwise engage with second end 103 of first connector 102 to allow fluid communication between first connector 102 and second connector 140 and the portions of tubing coupled thereto. As can be appreciated, first connector 102 and second connector 140 can be coupled and decoupled to permit fluid communication as desired. First connector 102 may detachably couple with second connector 140 to provide needle free connections. Advantageously, first connector 102 may pair with second connector 140 to form a leak-free closed system, allowing the delivery of various drugs or fluids.

In some embodiments, second connector 140 includes connecting portion 145 disposed opposite housing 144. Housing 144 may extend from connecting portion 145. Connecting portion 145 may include channel 149 disposed within connecting portion 145. Channel 149 may include opening 151, which may be disposed between first end 141 and second end 143. Opening 151 may be in fluid communication with channel 149 such that liquid entering opening 151 flows into channel 149 and through connecting portion 145. Channel 149 may also include outlet 155.

In some embodiments, connecting portion 145 includes outlet 155. Outlet 155 may be configured allow fluid to exit coupler assembly 100. In some embodiments, when first connector 102 is coupled to second connector 140, fluid enters first connector 102 via channel 116, flows through the fluid pathway formed between first connector 102 and second connector 140, and exits second connector 140 via outlet 155.

Connecting portion 145 may be configured to couple to a portion of tubing (e.g., tubing coupled to patient end 600) such that channel 149 is in fluid communication with the portion of tubing. In some embodiments, an internal surface of connecting portion 145 includes threads to couple or mate with the portion of tubing to allow second connector 140 to be in fluid communication with a portion of tubing.

Housing 144 may extend from connecting portion 145 towards first end 141. In some embodiments, channel 149 extends through housing 144. Housing 144 may include cavity 157 disposed within housing 144. Cavity 157 may be interior space within housing 144. In some embodiments, when second connector 140 is coupled to first connector 102, valve 104 is at least partially disposed within cavity 157 of second connector 140. This results in first connector 102 being in fluid communication with second connector 140 to form a fluid pathway between first connector 102 and second connector 140. When first connector 102 is coupled to second connector 140, valve 104 may be at least partially disposed within opening 151 such that valve 104 at least partially extends through opening 151 into cavity 157. In some embodiments, second connector 140 couples to mating portion 105 of first connector 102.

Referring to FIGS. 2-3, a fluid pathway is formed when coupler assembly 100 is in the first configuration (e.g., when first connector 102 is coupled to second connector 140). In the first configuration, fluid may flow into first connector 102 via channel 116 and into body 111. Fluid may then flow through fluid valve 104 into cavity 157. The fluid may then flow a through channel 149, out of second connector 140 via outlet 155.

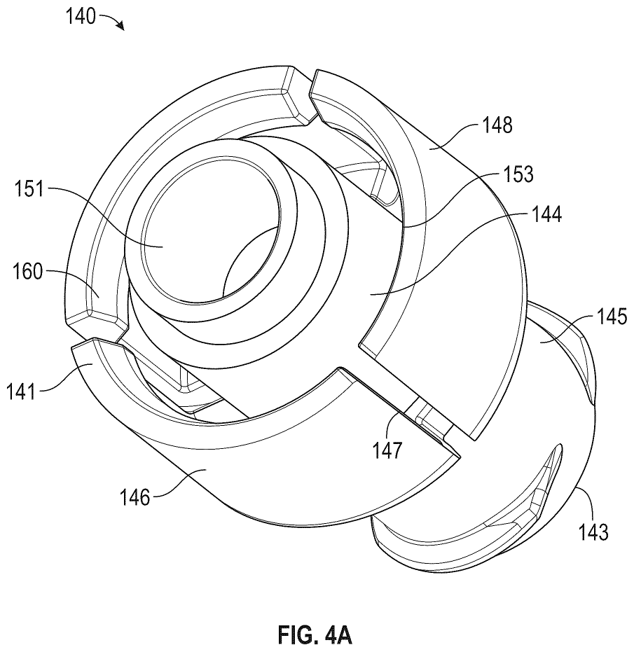

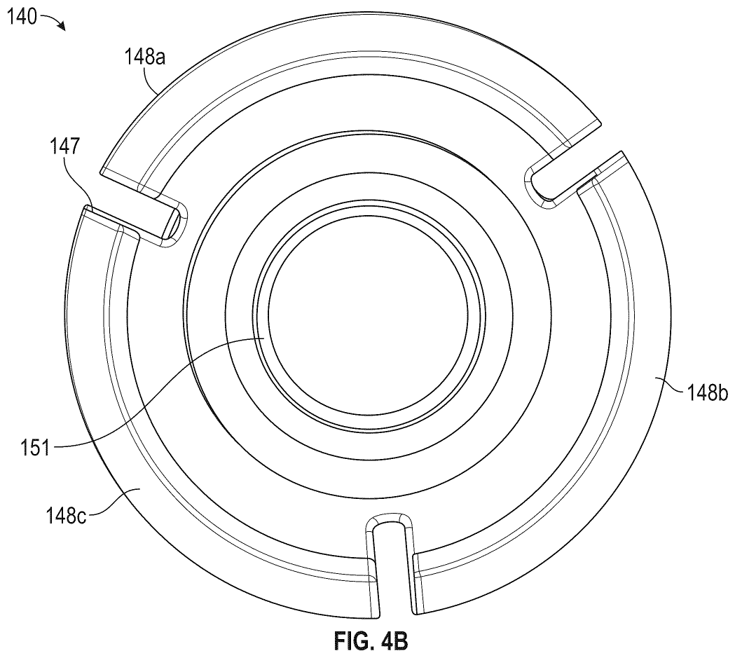

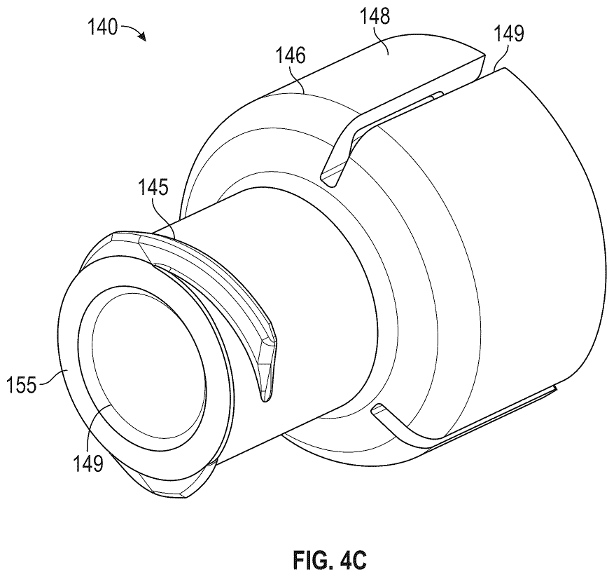

FIG. 4A is a front perspective view of a second connector of the coupler assembly of FIG. 1, in accordance with various aspects of the present disclosure. FIG. 4B is a front view of the second connector of FIG. 4A, in accordance with various aspects of the present disclosure. FIG. 4C is a back perspective view of the second connector of FIG. 4A, in accordance with various aspects of the present disclosure.

In some embodiments, second connector 140 is configured to couple and decouple form first connector 102. For example, when first connector 102 is coupled to second connector 140, second connector 140 may decouple from first connector 102 in response to a disconnection event. Second connector 140 may be configured to decouple from first connector 102 in response to a pullout force exceeding a predetermined threshold.

In some embodiments, second connector 140 includes ring 146. Ring 146 may extend outwardly from housing 144. Ring 146 may be a ring disposed around housing 144 (e.g., opening 151) and may be configured to engage with first connector 102 to couple to second connector 140 to first connector 102. Ring 146 may comprise the outer perimeter of second connector 140. In some embodiments, the diameter of ring 146 is the maximum diameter of second connector 140. In some embodiments, ring 146 is configured to removably couple to first connector 102. For example, ring 146 may allow second connector 140 to couple to first connector 102 and may allow second connector 140 to decouple from first connector 102 in response to a disconnection event.

In some embodiments, ring 146 is concentric with opening 151. For example, the center of ring 146 may be the center of opening 151. In some embodiments, when second connector 140 is coupled to first connector 102, central axis A-A extends through the center of opening 151 and ring 146. Ring 146 may have a diameter greater than the diameter of opening 151. In some embodiments, ring 146 is configured to engage with one or more grooves 109 of first connector 102 to couple second connector 140 to first connector 102.

In some embodiments, second connector 140 includes gap 153. Gap 153 may be the space between ring 146 and opening 151. In some embodiments, when first connector 102 is coupled to second connector 140, at least a portion of first connector 102 (e.g., mating portion 105) is disposed within gap 153.

In some embodiments, ring 146 includes one or more relief cuts or cuts 147. Cuts 147 may be configured to divide ring 146 into a plurality of flanges 148. For example, ring 146 may include three cuts 147 that divide ring 146 into three flanges 148a, 148b, and 148c. In some embodiments, ring 146 includes one, two, four, five, six, or greater than six cuts 147. Ring 146 may include a plurality of cuts 147 to divide sidewall into two, four, five, six or greater than six flanges 148. In some embodiments, cut 147 is configured to extend entirely through ring 146.

Referring to FIG. 4A, second connector 140 may include one or more ridges 160. Ridge 160 may extend from ring 146. In some embodiments, ridge 160 extends radially inward from ring 146. For example, ridge 160 may extend towards opening 151. Ridge 160 may be configured to engage with groove 109 to secure second connector 140 to first connector 102. In some embodiments, cuts 147 divide ridge 160 into a plurality of ridges 160, similar to flanges 148. Each flange 148 may include its own ridge 160 configured to engage with groove 109 of first connector 102.

In some embodiments, cuts 147 allow flanges 148 to move relative to each other. For example, cuts 147 may allow adjacent flanges 148 to flex and bend radially outward when second connector 140 is coupled and/or decoupled to and from first connector 102. In some embodiments, cuts 147 are configured to alleviate the straining of material during injection molding of second connector 140. For example, cuts 147 may allow for increase in tolerances when forming second connector 140. Cuts 147 may allow each flange 148 to move independently from one another (e.g., flex and deflect).

During coupling of second connector 140 to first connector 102, first connector 102 is inserted into gap 153 such ridges 160 of flanges 148 engage with grooves 109 of first connector 102. Flanges 148 may flex and bend radially outward due to cuts 147 to allow ridge 160 to engage with groove 109. Flanges 148 may be biased radially inward such that once ridge 160 is disposed within groove 108, flange 148 returns to its biased radially inward position to secure second connector 140 to first connector 102.

In some embodiments, cuts 147 are configured to allow second connector 140 to decouple from first connector 102 in response to a disconnection event (e.g., pullout force). For example, in response to a pullout force, flanges 148 may flex radially outward causing ridge 160 to no longer be disposed within groove 109 allowing second connector 140 to decouple from first connector 102. In some embodiments, cuts 147 have a width of 0.5 mm to 1 mm. Cuts 147 may have a width of approximately 0.25 mm to approximately 3.0 mm, approximately 0.5 mm to approximately 2.5 mm, approximately 0.75 mm to approximately 2.0 mm, approximately 1.0 mm to approximately 1.5 mm, greater than 3.0 mm or less than 0.25 mm. In some embodiments, flanges 148 is the weakest link of couple assembly 100. For example, flanges 148 may be the weakest coupling component of coupler assembly 100 allowing second connector 140 to decouple from first connector 102 via flanges 148.

In some embodiments, coupler assembly 100 is in the first configuration when flange 148 of second connector 140 is coupled to or disposed within groove 109 of mating portion 105. Ridge 160 may be disposed within groove 109, which prevents axial movement of second connector 140 relative to first connector 102 thereby securing second connector 140 to first connector 102.

Coupler assembly 100 may be configured to be in a second configuration. In the second configuration, second connector 140 is decoupled from first connector 102. In the second configuration, valve 104 closes and the fluid pathway between first connector 102 and second connector 140 is interrupted. In some embodiments, valve 104 returning to the closed position prevents flow of fluid out of first connector 102. Coupler assembly 100 may transition from the first configuration to the second configuration by second connector 140 decoupling from first connector 102.

In some embodiments, second connector 140 is configured to decouple from first connector 102 due to a disconnection event, which is caused by a pullout force. For example, a pullout force (e.g., force F) may be applied to second connector 140, either by being directly applied to second connector 140 or indirectly applied to second connector 140, such as being applied to tubing coupled to second connector 140 and/or first connector 102. The pullout force may cause second connector 140 to move axially away from first connector 102 along central axis A-A thereby decoupling second connector 140 from first connector 102.

In some embodiments, second connector 140 is decoupled from first connector 102 when force F exceeds a predetermined threshold force. For example, if force F is less than the predetermined threshold force, second connector 140 may not decouple from first connector 102. The predetermined threshold force prevents inadvertent or accidental decoupling based on minor forces or movements. The predetermined threshold force may be based on the flexibility and/or stiffness of flange 168 and/or ridge 160. For example, the higher the stiffness of flange 168, the higher the predetermined threshold force. In some embodiments, the number and/or the width of cuts 147 determines the predetermined threshold force. For example, an increase in the number of cuts 147 or increase in the width of cuts 147 may decrease the predetermined threshold force required. In some embodiments, an increase in the number of cuts 147 or increase in the width of cuts 147 reduces the contact area between second connector 140 and first connector 102 (e.g., ridge 160 and groove 109) thereby reducing the predetermined threshold force required to decouple second connector 140 form first connector 102.

In some embodiments, the predetermined threshold force is approximately 4 pounds (lbs). The predetermined threshold force may be from approximately 1 lb to approximately 8 lbs, approximately 3 lbs to approximately 7 lbs, approximately 4 lbs to approximately 6 lbs, or greater than 8 lbs. For example, a patient may have a needle/catheter inserted into their skin and the needle/catheter may be coupled to first connector 102 or second connector 140. The patient may walk away from an infusion pump or accidental pull on a fluid line coupled to first connector 102 or second connector 140 and the force exceeds 4 lbs, second connector 140 may automatically release or decouple from first connector 102, effectively closing the fluid pathway between first connector 102 and second connector 140, as described herein.

Second connector 140 may decouple from first connector 102 in response to the pullout force exceeding a predetermined threshold force. When the pullout force exceeds the predetermined threshold force, flanges 148 flex or bend radially outward due to ridge 160 being axially pulled out of and displaced from groove 109. For example, in response to the pullout force exceeding the predetermined threshold force, second connector 140 may axially move relative to first connector 102 such that flanges 168 flex or deflect radially outward causing ridge 160 from being displaced from groove 109 and second connector 140 no longer being secured to first connector 102.

In some embodiments, upon decoupling of first connector 102 from second connector 140, a user sterilizes first connector 102 and recouples first connector 102 to second connector 140. In some embodiments, a user may sterilize first connector 102 and/or second connector 140. First connector 102 may be recoupled to second connector 140 by inserting first connector 102 into second connector 140 (e.g., gap 153). First connector 102 may be axially moved relative to second connector 140 to couple first connector 102 to second connector 140 by engaging groove 109 with ridge 160 of flange 148 thereby securing second connector 140 to first connector 102. Recoupling first connector 102 to second connector 140 results in coupler assembly 100 transitioning from the second configuration to the first configuration.

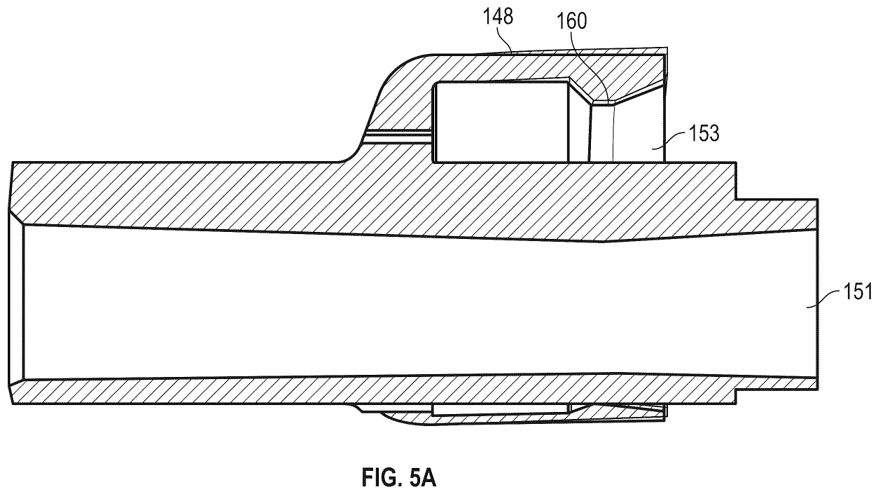

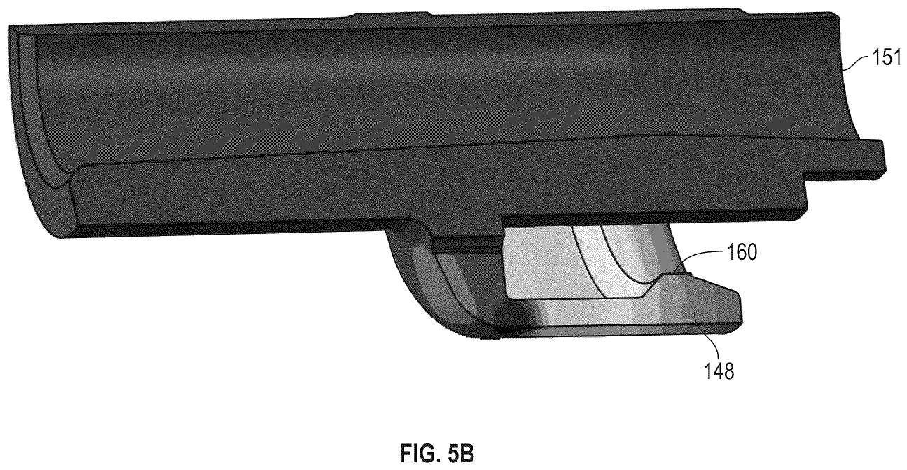

FIG. 5A is an illustration of a computer simulation showing the deformation of the second connector of FIG. 4, in accordance with various aspects of the present disclosure. FIG. 5B is an illustration of a computer simulation showing the deformation of the second connector of FIG. 4, in accordance with various aspects of the present disclosure.

In some embodiments, flanges 148 are configured to flex and bend causing strain to flanges 148. For example, flanges 148 may flex radially outwards away from their biased position of being radially inward. As shown in FIG. 5A, flange 148 may flex radially outward compared to its biased position shown in black wireframe. In its normal, unflexed position, flange 148 may be biased radially inward compared to when flange 148 is flexed radially outward. Flange 148 may be flexed radially outward to couple second connector 140 to first connector 102. Upon coupling of second connector 140 to first connector 102, flange 148 may return to its normal position of being biased radially inward (e.g., black wireframe of FIG. 5A). As shown in FIG. 5B, flange 148 is configured to flex and deform causing strain on flange 148. Flange 148, along with ring 146, may be comprised of a material allowing for flexing without deformation.

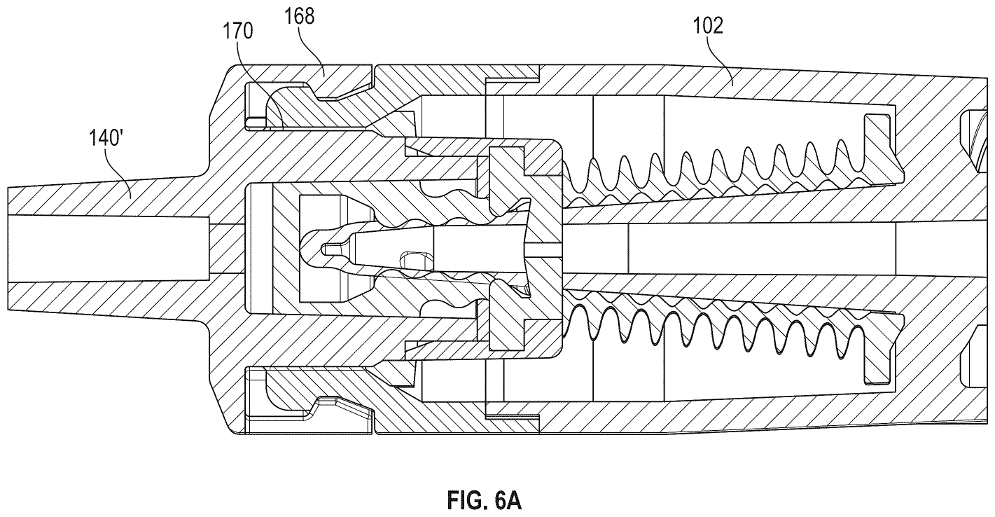

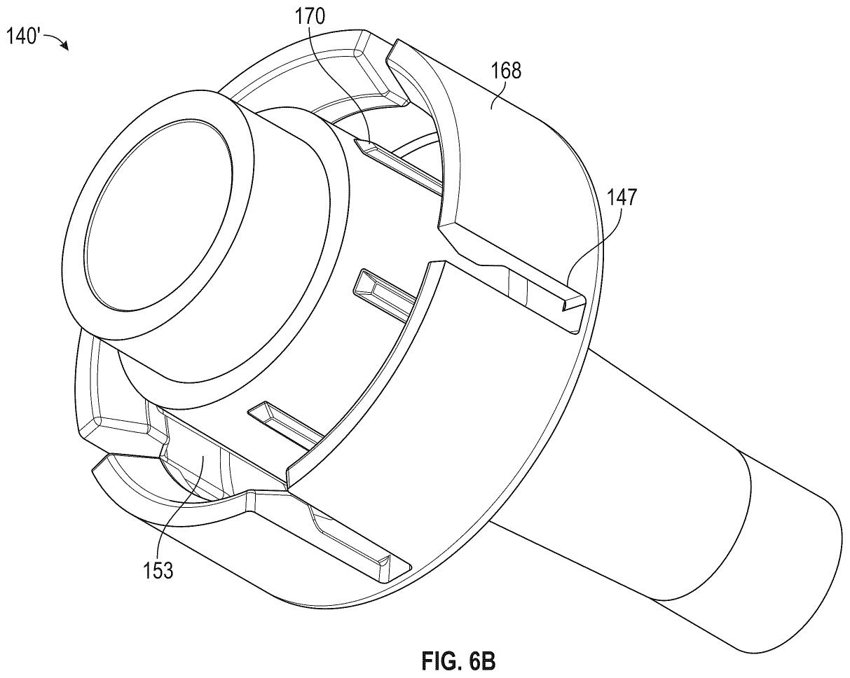

FIG. 6A is a zoomed in cross-sectional view of the coupler assembly of FIG. 1 having a second connector with centering ribs, in accordance with various aspects of the present disclosure. FIG. 6B is a side perspective view of the second connector of FIG. 6A, in accordance with various aspects of the present disclosure.

Referring to FIGS. 6A-6B, coupler assembly 100 may include second connector 140′. Second connector 140′ may be substantially similar to second connector 140, but second connector 140′ may include ribs 170. Ribs 170 may be configured to keep second connector 140 centered along central axis A-A and prevent slippage between ridge 160 and groove 109. Ribs 170 may be disposed on an outer perimeter of housing 144. In some embodiments, ribs 170 are circumferentially disposed around housing 144 such that ribs 170 are disposed within gap 153. Ribs 170 may be configured to decrease the contact surface area between mating portion 105 and housing 144 of second connector 140. In some embodiments, ribs 170 reduces the frictional force between mating portion 105 and housing 144 thereby allowing second connector 140 to easily decouple from first connector 102 in response to a disconnection event.

When second connector 140 is coupled to first connector 102, ribs 170 may engage with mating portion 105 of first connector 102. Ribs 170 may be configured to reduce the frictional force between mating portion 105 and housing 144 to allow first connector 102 to decouple from second connector 140 when the pull out force exceeds a predetermined amount. In some embodiments, ribs 170 are configured to secure second connector 140 in position (e.g., centered) relative to first connector 102 to prevent disruption of fluid flow from first connector 102 to second connector 140. In some embodiments, second connector 140′ having ribs 170 decrease the pullout force required to decouple second connector 140 from first connector 102.

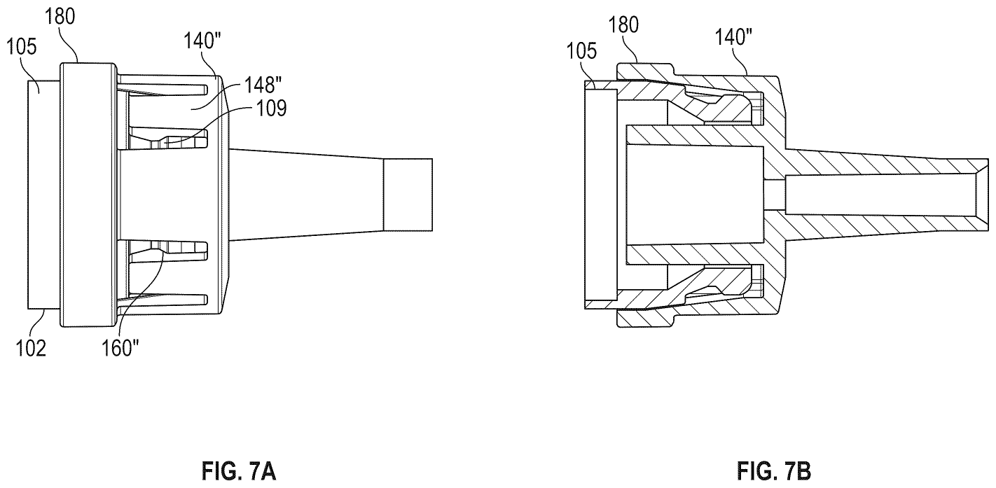

FIG. 7A is a side view of a second connector of having a bearing ring, in accordance with various aspects of the present disclosure. FIG. 7B is a cross-sectional side view of the second connector of FIG. 7A, in accordance with various aspects of the present disclosure.

Referring to FIGS. 7A-7B, coupler assembly 100 may include second connector 140″. Second connector 140″ may be substantially similar to second connector 140′ and second connector 140, but second connector 140″ may include ring 180. Ring 180 may be configured to create a bearing surface between groove 109 and ridge 160″/flange 148″. For example, similar to second connector 140 and 140″, second connector 140″ may include flange 148″ having ridge 160″ configured to engage with groove 109 of mating portion 105. Ring 180 may be configured to assist in securing first connector 102 to second connector 140″ when second connector 140″ is coupled to first connector 102. For example, ring 180 may be configured to stabilize first connector 102 and second connector 140″ together when second connector 140″ is coupled to first connector 102. In some embodiments, ring 180 is configured to abut mating portion 105 as ridge 160″ is disposed within or proximate groove 109. For example, ring 180 may be circumferentially disposed around mating portion 105 when second connector 140″ is coupled to first connector 102. In some embodiments, flange 148″ does not include ridge 160″ and includes ring 180 to assist in securing mating portion 105 to second connector 140.

The disclosures described herein include at least the following clauses:

Clause 1: A coupler comprising a first connector having a first end, a second end opposite the first end, and a valve disposed between the first end and the second end, the second end including a mating portion, wherein the valve extends at least partially into the mating portion, a second connector having a housing, a plurality of flanges extending from the housing, each of the plurality of flanges being separated from an adjacent flange by a cut, the plurality of flanges configured to engage with the mating portion when the second connector is coupled to the first connector. The first connector is configured to decouple from the second connector in response to a pullout force exceeding a predetermined threshold force.

Clause 2: The coupler of clause 1, wherein the mating portion includes a groove circumferentially disposed on the mating portion, the groove configured to receive a portion of one of the plurality of flanges.

Clause 3: The coupler of clause 2, wherein each of the plurality of flanges includes a ridge, the ridge configured to engage the groove when the first connector is coupled to the second connector.

Clause 4: The coupler of clause 1, wherein the second connector includes three cuts and three flanges.

Clause 5: The coupler of clause 1, wherein the pullout force is a force applied to the second connector along a central axis of the second connector and the central axis extends at least along a length of the second connector.

Clause 6: The coupler of clause 5, wherein the central axis extends through the first connector and the second connector when the first connector is coupled to the second connector.

Clause 7: The coupler of clause 1, wherein the valve is configured to extend at least partially into the housing when the first connector is coupled to the second connector.

Clause 8: The coupler of clause 1, wherein the coupler has a first configuration and in the first configuration the first connector is coupled to the second connector such that the valve is at least partially disposed within the housing.

Clause 9: The coupler of clause 1, wherein the coupler has a second configuration and in the second configuration the first connector is disconnected from the second connector.

Clause 10: The coupler of clause 1, wherein the first connector is coupled to a first portion of tubing at the first end and the second connector is coupled to a second portion of tubing at the connecting portion.

Clause 11: The coupler of clause 1, wherein the second connector includes a gap disposed between the plurality of flanges and the housing and the mating portion is at least partially disposed within the gap when the first connector is coupled to the second connector.

Clause 12: The coupler of clause 1, wherein a fluid pathway is formed between the second connector and the first connector when the first connector is coupled to the second connector.

Clause 13: The coupler of clause 1, wherein the housing includes a plurality of ribs circumferentially disposed around the housing, the plurality of ribs configured to contact the mating portion when the first connector is coupled to the second connector.

Clause 14: The coupler of clause 1, wherein the housing includes a ring circumferentially disposed around the housing, the ring configured to contact the mating portion when the first connector is coupled to the second connector.

Clause 15: The coupler of clause 1, wherein the plurality of flanges are biased radially inward and are configured to flex radially outward when coupling the first connector to the second connector.

Clause 16: The coupler of clause 1, wherein the plurality of flanges form a ring, the ring being concentric with an opening of the housing.

Clause 17: The coupler of clause 1, wherein the plurality of flanges are configured to deflect away from a central axis when decoupling the first connector to the second connector.

Clause 18: The coupler of clause 1, wherein the mating portion is at least partially disposed within the second connector when the first connector is coupled to the second connector.

Clause 19: A coupler comprising a first connector having a first end, a second end opposite the first end, a mating portion disposed proximate the second end, the first connector including a valve disposed between the first end and the second end, the valve at least partially extending into the mating portion, and a second connector having a housing and ring extending from the housing, the ring having a plurality of cuts forming a plurality of flanges, each of the plurality of flanges including a ridge extending radially inward and each of the plurality of flanges being biased radially inward. The first connector is configured to decouple from the second connector in response to a pullout force exceeding a predetermined threshold force. At least one of the plurality of flanges is configured to deflect radially outward to allow the first connector to decouple from the second connector when the pullout force exceeds the predetermined threshold force.

Clause 20: A coupler comprising a first connector having a first end, a second end opposite the first end, a tubing portion disposed proximate the first end and a mating portion disposed proximate the second end, the first connector including a valve disposed between the first end and the second end, the valve at least partially extending into the mating portion, the first connector including a groove circumferentially disposed around the mating portion, and a second connector having a housing and ring extending from the housing, the ring having a plurality of cuts forming a plurality of flanges, each of the plurality of flanges including a ridge extending radially inward and each of the plurality of flanges being biased radially inward, the second connector including a gap between the housing and the ring, the gap configured to receive a portion of the mating portion when the first connector is coupled to the second connector. A fluid pathway is formed between the first connector and the second connector when the first connector is coupled to the second connector. The first connector is configured to decouple from the second connector in response to a pullout force exceeding a predetermined threshold force. At least one of the plurality of flanges is configured to deflect radially outward to allow the first connector to decouple from the second connector when the pullout force exceeds the predetermined threshold force.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A coupler comprising:
a first connector having a first end, a second end opposite the first end, and a valve disposed between the first end and the second end, the second end including a mating portion, wherein the valve extends at least partially into the mating portion; and
a second connector having a housing, a plurality of flanges extending from the housing, each of the plurality of flanges being separated from an adjacent flange by a cut, the plurality of flanges configured to engage with the mating portion when the second connector is coupled to the first connector,
wherein the first connector is configured to decouple from the second connector in response to a pullout force exceeding a predetermined threshold force.

2. The coupler of claim 1, wherein the mating portion includes a groove circumferentially disposed on the mating portion, the groove configured to receive a portion of one of the plurality of flanges.

3. The coupler of claim 2, wherein each of the plurality of flanges includes a ridge, the ridge configured to engage the groove when the first connector is coupled to the second connector.

4. The coupler of claim 1, wherein the second connector includes three cuts and three flanges.

5. The coupler of claim 1, wherein the pullout force is a force applied to the second connector along a central axis of the second connector and the central axis extends at least along a length of the second connector.

6. The coupler of claim 5, wherein the central axis extends through the first connector and the second connector when the first connector is coupled to the second connector.

7. The coupler of claim 1, wherein the valve is configured to extend at least partially into the housing when the first connector is coupled to the second connector.

8. The coupler of claim 1, wherein the coupler has a first configuration and in the first configuration the first connector is coupled to the second connector such that the valve is at least partially disposed within the housing.

9. The coupler of claim 1, wherein the coupler has a second configuration and in the second configuration the first connector is disconnected from the second connector.

10. The coupler of claim 1, wherein the first connector is coupled to a first portion of tubing at the first end and the second connector is coupled to a second portion of tubing at the connecting portion.

11. The coupler of claim 1, wherein the second connector includes a gap disposed between the plurality of flanges and the housing and the mating portion is at least partially disposed within the gap when the first connector is coupled to the second connector.

12. The coupler of claim 1, wherein a fluid pathway is formed between the second connector and the first connector when the first connector is coupled to the second connector.

13. The coupler of claim 1, wherein the housing includes a plurality of ribs circumferentially disposed around the housing, the plurality of ribs configured to contact the mating portion when the first connector is coupled to the second connector.

14. The coupler of claim 1, wherein the housing includes a ring circumferentially disposed around the housing, the ring configured to contact the mating portion when the first connector is coupled to the second connector.

15. The coupler of claim 1, wherein the plurality of flanges are biased radially inward and are configured to flex radially outward when coupling the first connector to the second connector.

16. The coupler of claim 1, wherein the plurality of flanges form a ring, the ring being concentric with an opening of the housing.

17. The coupler of claim 1, wherein the plurality of flanges are configured to deflect away from a central axis when decoupling the first connector to the second connector.

18. The coupler of claim 1, wherein the mating portion is at least partially disposed within the second connector when the first connector is coupled to the second connector.

19. A coupler comprising:
a first connector having a first end, a second end opposite the first end, a mating portion disposed proximate the second end, the first connector including a valve disposed between the first end and the second end, the valve at least partially extending into the mating portion; and
a second connector having a housing and ring extending from the housing, the ring having a plurality of cuts forming a plurality of flanges, each of the plurality of flanges including a ridge extending radially inward and each of the plurality of flanges being biased radially inward,
wherein the first connector is configured to decouple from the second connector in response to a pullout force exceeding a predetermined threshold force,
wherein at least one of the plurality of flanges is configured to deflect radially outward to allow the first connector to decouple from the second connector when the pullout force exceeds the predetermined threshold force.

20. A coupler comprising:
a first connector having a first end, a second end opposite the first end, a tubing portion disposed proximate the first end and a mating portion disposed proximate the second end, the first connector including a valve disposed between the first end and the second end, the valve at least partially extending into the mating portion, the first connector including a groove circumferentially disposed around the mating portion; and
a second connector having a housing and ring extending from the housing, the ring having a plurality of cuts forming a plurality of flanges, each of the plurality of flanges including a ridge extending radially inward and each of the plurality of flanges being biased radially inward, the second connector including a gap between the housing and the ring, the gap configured to receive a portion of the mating portion when the first connector is coupled to the second connector,
wherein a fluid pathway is formed between the first connector and the second connector when the first connector is coupled to the second connector,
wherein the first connector is configured to decouple from the second connector in response to a pullout force exceeding a predetermined threshold force,
wherein at least one of the plurality of flanges is configured to deflect radially outward to allow the first connector to decouple from the second connector when the pullout force exceeds the predetermined threshold force.
